(19) United States
(12) Patent Application Publication
(10) Pub. No.: US 2018/0078529 A1
(43) Pub. Date: Mar. 22, 2018

(54) ANGIOTENSIN II RECEPTOR AGONIST FOR TREATING PULMONARY FIBROSIS

(71) Applicant: VICORE PHARMA AB, Mölndal (SE)

(72) Inventors: Björn DAHLÖF, Landvetter (SE); Anders LJUNGGREN, Mölndal (SE)

(21) Appl. No.: 15/554,213

(22) PCT Filed: Mar. 2, 2016
(86) PCT No.: PCT/GB2016/050552

§ 371 Date: Aug. 28, 2017

Related U.S. Application Data
(60) Provisional application No. 62/126,880, filed on Mar. 2, 2015.

(51) Int. Cl. A61K 31/4178; A61K 9/00; A61K 45/06

(52) U.S. Cl. CPC A61K 31/4178; A61K 45/06; A61K 9/0053; A61K 9/0073

(57) ABSTRACT

This invention relates to a new use of compounds that are angiotensin II (Ang II) receptor agonists, more particularly agonists of the Ang II type 2 receptor (the AT2 receptor), and especially agonists that bind selectively to the AT2 receptor, for therapeutic treatment of pulmonary fibrosis, in particular idiopathic pulmonary fibrosis.

### FIELD OF THE INVENTION

This invention relates to a new use of compounds that are angiotensin II (Ang II) receptor agonists, more particularly selective agonists of the Ang II type 2 receptor (hereinafter the AT2 receptor), and especially agonists that bind selectively to that receptor, for therapeutic treatment of pulmonary fibrosis, in particular idiopathic pulmonary fibrosis.

### BACKGROUND OF THE INVENTION

Idiopathic pulmonary fibrosis (IPF) is a lung-disease of unknown cause with no curative treatment options except in rare cases lung transplantation, resulting in a chronic, irreversible, progressive deterioration in lung function and, in most cases, leading to death within 2-5 years (median survival 2.5 to 3.5 years). While the overall prognosis is poor in IPF, it is difficult to predict the rate of progression in individual patients. Risk factors for IPF include age, male gender, genetic predisposition and history of cigarette smoking. The annual incidence is between 5-16 per 100,000 individuals, with a prevalence of 13-20 cases per 100,000 people, increasing dramatically with age (King Jr T E et al. Lancet 2011; 378:1949-1961, Noble P W et al. J Clin Invest 2012; 122:2756-2762). IPF is limited to the lungs and is recalcitrant to therapies that targets the immune system which distinguishes it from pulmonary fibrosis associated with systemic diseases (Noble P W et al. 2012)

Patients with IPF usually seek medical assistance due to chronic and progressive exertional dyspnea and cough. Imaging of the lung classically reveals traction bronchiectasis, thickened interlobar septae and subpleural honeycombing. When all three manifestations are present and there is no evidence of a systemic connective tissue disease or environmental exposure, a diagnosis of IPF is very likely. A definite diagnosis is usually made by lung biopsy and requires a multidisciplinary team of expertise including pulmonologists, radiologists and pathologists experienced in interstitial lung diseases (King Jr T E et al. 2011). IPF demonstrates different phenotypes with different prognosis, defined as mild, moderate and severe. Mild cases follow a stable or slow progressive path with patients sometimes taking several years to seek medical advice. Accelerated IPF has a much more rapid progression with shortened survival, affecting a sub-group of patients, usually male cigarette smokers. Acute exacerbations of IPF are defined as a rapid worsening of the disease, and patients in this sub-population have very poor outcomes with a high mortality rate in the short run (King Jr T E et al. 2011). The cause of IPF is unknown but it appears to be a disorder likely arising from an interplay of environmental and genetic factors resulting in fibroblast driven unrelenting tissue remodeling rather than normal repair; a pathogenesis primarily fibrosis driven rather than inflammatory driven (Noble P W et al. 2012). A growing body of evidence suggests that the disease is initiated through alveolar epithelial cell microinjuries and apoptosis, activating neighboring epithelial cells and attracting stem or progenitor cells that produce the factors responsible for the expansion of the fibroblast and myofibroblast populations in a tumour like way. The fibroblastic foci secrete exaggerated amounts of extracellular matrix that destroys the lung parenchyma and ultimately leads to loss of lung function (King Jr T E et al. 2011).

The mean annual rate of decline in lung function is within a range of 0.13-0.21 litres. Symptoms precede diagnosis by 1-2 years and radiographic signs may precede symptoms (Ley B et al. Am J Respir Crit Care Med 2011; 183:431-440).

Numerous treatment approaches have been tested in pre-clinical models and clinical trials such as anti-inflammatory, immune-modulatory, cytotoxic, general anti-fibrotic, anti-oxidant, anti-coagulant, anti-chemochine, anti-angiogenic drugs as well as Renin-Angiotensin System (RAS)-blockers, endothelin antagonists, Sildenafil and Thalidomide and basically been shown to provide limited or no benefits (Rafii Ret al. J Thorac Dis 2013; 5, 48-73),

In late clinical stage, Nintedanib has promising results on decline in lung function but no effect on mortality and limiting side effects such frequent diarrhea (Richeldi L et al NEJM 2014, online May 18). The only drug approved for clinical use in treating IPF worldwide is Pirfenidone. Pirfenidone is anti-inflammatory and an antioxidant, which also works as an antifibrotic. It has significant effects on lung capacity and exercise tolerance in milder forms of IPF and some effect on mortality with some associated side effects (King Jr T E et al NEJM 2014, online May 18).

In general, IPF patients with a moderate-to-severe functional lung impairment and associated co-morbidities (e.g. pulmonary hypertension) have been excluded from clinical trials, which are mainly performed on IPF patients with mild-to-moderate diagnosis. To restore the alveolar epithelium is very desirable as a therapeutic effect in IPF, and therefore stem cell therapy has also been tested. Some preclinical studies have shown promise in the use of pluripotent stem cells that can differentiate into lung epithelial and endothelial cells, thereby repairing lung injury and fibrosis.

Currently, a lung transplant is the only intervention that substantially improves survival in IPF patients: however, complications such as infections and transplant rejection are not un-common (King Jr T E et al. 2011).

The development of new treatment strategies for IPF is therefore important. Thus, the fundamental challenge for the future is to develop appropriate therapeutic approaches that will reverse or stop the progression of the disease.

### SUMMARY OF THE INVENTION

Compounds of the invention are agonists of Ang II receptor, more particularly, are agonists of the AT2 receptor, and, especially, are selective agonists of that sub-receptor, in particular non-peptide selective agonists. In some embodiments, the compounds of the invention are those that can stimulate AT2 receptors.

Compounds of the invention can therefore be used for the therapeutic treatment of pulmonary fibrosis, in particular IPF. In some embodiments, a compound of the invention can be N-Butyloxycarbonyl-3-(4-imidazol-1-ylmethylphenyl)-5-iso-butylthiophene-2-sulfonamide (Compound 21 or in short C21). Pharmaceutically acceptable salts, solvates and prodrugs of C21 are also useful for the therapeutic treatment of pulmonary fibrosis, in particular IPF.

In one embodiment of the present invention, there is provided a method of therapeutic treatment of pulmonary fibrosis, in particular IPF, which method comprises administration of a therapeutically effective amount of a compound of the invention (or a pharmaceutically acceptable salt, solvate or prodrug thereof) to a person suffering from pulmonary fibrosis, in particular idiopathic pulmonary fibrosis (IPF).

In another embodiment of the invention, compounds of the invention (e.g., AT2 receptor agonists or compounds that stimulate AT2 receptors, in particular non-peptide agonists), or a pharmaceutically acceptable salt, solvate or prodrug thereof, may also be used in the manufacture of a medicament for the treatment of idiopathic pulmonary fibrosis. In some embodiments, the compound can be N-Butyloxycarbonyl-3-(4-imidazol-1-ylmethylphenyl)-5-iso-butylthiophene-2-sulfonamide (C21), as well as pharmaceutically acceptable salts, solvates or prodrugs thereof, which may be used in the manufacture of a medicament for the treatment of idiopathic pulmonary fibrosis.

According to a further embodiment of the invention, there is provided a method of treating pulmonary fibrosis, in particular idiopathic pulmonary fibrosis, which method comprises administering a compound of the invention (e.g., an AT2 receptor agonist or other compound that stimulates an AT2 receptor in particular non-peptide agonists), or a pharmaceutically acceptable salt, solvate or prodrug thereof, to a patient in need of such therapy. In some embodiments, a method of treating pulmonary fibrosis, in particular idiopathic pulmonary fibrosis, comprises administering the compound N-Butyloxycarbonyl-3-(4-imidazol-1-ylmethylphenyl)-5-iso-butylthiophene-2-sulfonamide (C21), or a pharmaceutically acceptable salt, solvate or prodrug thereof, to a patient in need of such therapy.

In some embodiments, there is provided a method of treating pulmonary fibrosis, in particular idiopathic pulmonary fibrosis, comprising administrating a therapeutically effective amount of a compound of the invention (e.g., an AT2 receptor agonist or other compound that stimulates an AT2 receptor in particular non-peptide agonists), or a pharmaceutically acceptable salt, solvate or prodrug thereof, through a combination of administrative routes, either separately, sequentially or in parallel at the same time (e.g., concurrently), preferably via inhalation and orally, in order to achieve effective amount or dosage, to a patient in need of such a therapy.

In some embodiments, there is provided a method of treating pulmonary fibrosis, in particular idiopathic pulmonary fibrosis, comprising administrating a therapeutically effective amount of the compound N-Butyloxycarbonyl-3-(4-imidazol-1-ylmethylphenyl)-5-iso-butylthiophene-2-sulfonamide (C21), or a pharmaceutically acceptable salt, solvate or prodrug thereof, through a combination of administrative routes, either separately, sequentially or in parallel at the same time (e.g., concurrently), preferably via inhalation and orally, in order to achieve effective dosage, to a patient in need of such a therapy.

Other embodiments and advantages will be more fully apparent from the following disclosure.

### BRIEF DESCRIPTION OF THE DRAWINGS

The following drawings are provided to illustrate various aspects of the present inventive concept and are not intended to limit the scope of the present invention unless specified herein.

FIG. 1 presents the structure of Compound 21 or in short C21, N-Butyloxycarbonyl-3-(4-imidazol-1-ylmethylphenyl)-5-iso-butylthiophene-2-sulfonamide.

### DETAILED DESCRIPTION

The foregoing and other aspects of the present invention will now be described in more detail with respect to the description and methodologies provided herein. It should be appreciated that the invention may be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention.

As used in the description of the embodiments of the invention, the singular forms “a”, “an” and “the” are intended to include the plural forms as well, unless the context clearly indicates otherwise. Also, as used herein, “and/or” refers to and encompasses any and all possible combinations of one or more of the associated listed items.

Furthermore, the term “about,” as used herein when referring to a measurable value such as an amount of a compound, dose, time, temperature, and the like, refers to variations of 20%, 10%, 5%, 1%, 0.5%, or even 0.1% of the specified amount.

When a range is employed (e.g., a range from x to y) it is it meant that the measurable value is a range from about x to about y, or any range therein, such as about x₁ to about y₁, etc.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms, including technical and scientific terms used in the description, have the same meaning as commonly understood by one of ordinary skill in the art to which this invention belongs.

“Effective amount” or dosage as used herein refers to an amount of a compound, composition and/or formulation of the invention that is sufficient to produce a desired effect, which can be a therapeutic and/or beneficial effect. The effective amount or dosage will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” or dosage in any individual case can be determined by one of skill in the art by reference to the pertinent texts and literature and/or by using routine experimentation.

By the term “treat,” “treating,” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

A “therapeutically effective” amount as used herein is an amount that is sufficient to treat (as defined herein) the subject. Those skilled in the art will appreciate that the therapeutic effects need not be complete or curative, as long as some benefit is provided to the subject.

A “subject in need” of the methods of the invention can be a subject known to have or suspected of having pulmonary fibrosis.

As used herein the term “concomitant administration” or “combination administration” of a compound, therapeutic agent or known drug with a compound of the present invention means administration of a known medication or drug and, in addition, the one or more compounds of the invention at such time that both the known drug and the compound will have a therapeutic effect. In some cases, this therapeutic effect will be synergistic. Such concomitant administration can involve concurrent (i.e., at the same time, in parallel at the same time), prior, or subsequent administration (e.g., sequential) of the known drug with respect to the administration of a compound of the present invention. Such concomitant or combination administration may also refer to administration of a compound of the invention through different administrative routes separately (e.g., at least about 2 or more hours apart), sequentially (e.g., within about 2 hours, e.g., about 15 sec, 30 sec, 45 sec, 1 min, 2, min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, and the like, and any range or value therein) and/or in parallel at the same time (e.g., concurrently) in order to achieve effective amount or dosage. A person of skill in the art, would have no difficulty determining the appropriate timing, sequence and dosages of administration for particular drugs and compounds of the present invention.

In addition, in some embodiments, the compounds of this invention will be used, either alone or in combination with each other or in combination with one or more other therapeutic medications as described herein, or their pharmaceutically acceptable salts, solvates or prodrugs, for manufacturing a medicament for the purpose of providing treatment for pulmonary fibrosis, in particular for treatment of idiopathic pulmonary fibrosis.

All patents, patent applications and publications referred to herein are incorporated by reference in their entirety. In the event of conflicting terminology, the present specification is controlling. Further, the embodiments described in one aspect of the present invention are not limited to the aspect described. The embodiments may also be applied to a different aspect of the invention as long as the embodiments do not prevent these aspects of the invention from operating for their intended purpose.

The RAS is a key regulator of blood pressure homeostasis. Renin, a protease, cleaves its only known substrate (angiotensinogen) to form angiotensin I, which in turn serves as substrate to angiotensin converting enzyme (ACE) to form Ang II. The endogenous hormone Ang II is a linear octapeptide (Asp¹-Arg²-Val³-Tyr⁴-Ile⁵-His⁶-Pro⁷-Phe⁸), and is an active component of the RAS. Renin and Ang II have both been implicated in IPF pathogenesis. Ang II is a powerful vasoactive hormone whose pleiotropic effects are mediated by two receptors highly expressed in IPF lungs: AT1 and AT2. Ang II induces apoptosis in alveolar epithelial cells and pulmonary arterial endothelial cells and the proliferation, activation, and migration of fibroblasts, resulting in abnormal deposition of extra cellular matrix components. The AT1 receptor is expressed in most organs, and is believed to be responsible for the majority of the pathological effects of Ang II. The safety and efficacy of losartan (an AT1-receptor inhibitor) are currently being investigated in a phase II open-label clinical trial of IPF (www.clinicaltrials.gov identifier NCT00879879).

Several studies in adult individuals appear to demonstrate that, in the modulation of the response following Ang II stimulation, activation of the AT2 receptor has opposing effects to those mediated by the AT1 receptor.

The AT2 receptor has also been shown to be involved in apoptosis and inhibition of cell proliferation (de Gasparo M et al. Pharmacol Rev 2000; 52:415-472). More recently, AT2 receptor agonists have been shown to be of potential utility in the treatment and/or prophylaxis of disorders of the alimentary tract, such as dyspepsia and irritable bowel syndrome, as well as multiple organ failure (see international patent application WO 99/43339).

The expected pharmacological effects of agonism of the AT2 receptor are described in general in de Gasparo M et al., 2000. It is not mentioned that agonism of the AT2 receptor may be used to treat IPF.

Recent studies indicate that Ang II may contribute to pulmonary fibrosis progression. Effects of Ang II on cell growth, inflammation and extracellular matrix synthesis are mainly coupled to AT1, whereas the function of AT2 has been heavily investigated and new research indicates that it is more prevalent in damaged tissue and exerts reparative properties. The expression level of the AT-receptors is changed in favor of the AT2 receptor expression during lung fibrosis development (Parra E R, et al. Clinics 2014; 69:47-54.). The balance of AT1 and AT2 receptors and lymphatic vessels modulate lung remodeling and fibrosis in systemic sclerosis and IPF, thereby changing the cellular response to Ang II.

AT2 receptor agonists have also been described in the prior art, for instance in international patent application WO 2002/096883. However, the use of those compounds in the treatment of IPF is not mentioned.

The listing or discussion of an apparently prior-published document in this specification should not necessarily be taken as an acknowledgement that the document is part of the state of the art or is common general knowledge.

According to a first aspect of the invention, there is provided an AT2 receptor agonist, or a compound that stimulates AT2 receptors, or a non-peptide AT2 receptor agonist, or a pharmaceutically acceptable salt (preferably the HCl salt of the compound of the invention), solvate or prodrug thereof, for use in the therapeutic treatment of pulmonary fibrosis, in particular IPF. Such AT2 receptor agonists and/or compounds that stimulate AT2 receptors may be referred to herein as the “compounds of the invention”. In some embodiments, a compound of the invention can be N-Butyloxycarbonyl-3-(4-imidazol-1-ylmethylphenyl)-5-iso-butylthiophene-2-sulfonamide (C21).

Thus, a compound of the invention includes AT2 receptor agonists that fully and those that partially activate the AT2 receptor and those compounds that can stimulate or activate the AT2 receptor. In some embodiments, an AT2 receptor agonist may be defined to include any compound that can stimulate or activate the AT2 receptor. In some embodiments, the compound of the invention is an AT2 receptor specific agonist and binds selectively to the AT2 receptor. In some embodiments, the compound of invention is a non-peptide AT2 receptor specific agonist that binds selectively to the AT2 receptor.

Non-limiting examples of compounds of the invention include N-Butyloxycarbonyl-3-(4-imidazol-1-ylmethylphenyl)-5-iso-butylthiophene-2-sulfonamide (C21).

Non-limiting examples of compounds of the invention that bind selectively to the AT2 receptor include N-Butyloxycarbonyl-3-(4-imidazol-1-ylmethylphenyl)-5-iso-butylthiophene-2-sulfonamide (C21).

According to a second aspect of the invention, there is provided the use of a compound of the invention, in particular the compound N-Butyloxycarbonyl-3-(4-imidazol-1-ylmethylphenyl)-5-iso-butylthiophene-2-sulfonamide (C21) or a pharmaceutically acceptable salt (preferably an HCl salt of the compound of the invention), solvate or prodrug thereof, for the therapeutic treatment of pulmonary fibrosis, in particular IPF.

In another aspect of the invention, the compounds of the invention (e.g., AT2 receptor agonists, and other compounds that stimulate AT2 receptors), or a pharmaceutically acceptable salt (e.g., an HCl salt of the compound of the invention), solvates or prodrug thereof, may also be used in the manufacture of a medicament for the therapeutic treatment of pulmonary fibrosis, in particular IPF.

According to a third aspect of the invention, there is provided a method of treating pulmonary fibrosis, in particular idiopathic pulmonary fibrosis, which method comprises administering a compound of the invention or a pharmaceutically acceptable salt, solvate or prodrug thereof, to a patient in need of such therapy. In some embodiments, the compound of the invention can be N-Butyloxycarbonyl-3-(4-imidazol-1-ylmethylphenyl)-5-iso-butylthiophene-2-sulfonamide (C21), or a pharmaceutically acceptable salt (preferably an HCl salt of the compound of the invention), solvate or prodrug thereof.

Pharmaceutically-acceptable salts include, but are not limited to, acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo or by freeze-drying). Salts may also be prepared by exchanging a counter-ion of a compound of the invention in the form of a salt with another counter-ion, for example using a suitable ion exchange resin. For the avoidance of doubt, other pharmaceutically acceptable derivatives of compounds of the invention are included within the scope of the invention (e.g. solvates, prodrugs etc).

As used herein, a “prodrug” is a composition that undergoes an in vivo modification when administered to a subject, wherein the product of the in vivo modification is a therapeutically effective compound. Prodrugs of compounds may be prepared by, for example, preparing a given compound as an ester. Thus, for example, an esterified form of the compound may be administered to a subject and may be de-esterified in vivo thereby releasing a therapeutically effective compound. Alternatively, some compounds may be prepared as prodrugs by adding short polypeptides (e.g., 1-6 amino acids) to the compound. Such prodrugs when administered to a subject may be cleaved (by, e.g., trypsin or other peptidases/proteases) thereby releasing a therapeutically effective compound. Formation of prodrugs is not limited by the specific examples described herein. Other ways of preparing therapeutically effective compounds as prodrugs are known.

Compounds of the invention may exhibit tautomerism. All tautomeric forms and mixtures thereof are included within the scope of the invention.

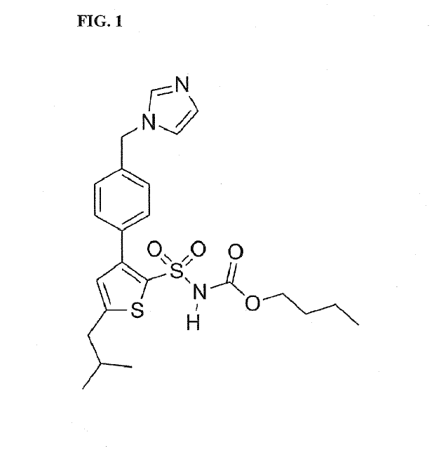

Compounds of the invention also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the invention. The compound N-Butyloxycarbonyl-3-(4-imidazol-1-ylmethylphenyl)-5-iso-butylthiophene-2-sulfonamide (C21) with the structure provided in FIG. 1, may be made in accordance with techniques well known to those skilled in the art, for example as described in international patent application WO 2002/096883, and all of its content is hereby incorporated by reference.

The compounds of the invention are useful because they possess pharmacological activity. In particular, the compounds of the invention are agonists of Ang II receptor, more particularly, they are agonists of the AT2 receptor, and, especially, are selective agonists of that sub-receptor. Compounds of the invention have the advantage that they bind selectively to, and exhibit agonist activity at, the AT2 receptor. By compounds that “bind selectively” to the AT2 receptor, we include that the affinity ratio for the relevant compound (AT2:AT1) is at least 100:1, preferably at least 1000:1, more preferably at least 10000:1, and even more preferably at least 25000:1.

It has now been found that compounds of the invention (e.g., AT2 receptor agonists) are useful in the treatment of pulmonary fibrosis, in particular IPF. According to a further aspect of the present invention, there is provided a method of treatment of pulmonary fibrosis, in particular IPF, which method comprises administration of a therapeutically effective amount of a compound of the invention (or a pharmaceutically acceptable salt thereof) to a person suffering from, such a condition.

The compounds of the invention will normally be administered orally, intravenously, subcutaneously, buccally, rectally, dermally, nasally, tracheally, bronchially, by any other parenteral route, or via inhalation or pulmonary route, or any combination thereof, in a pharmaceutically acceptable dosage form, in solution, in suspension, including nanosuspensions, or in liposome formulation. Additional methods of administration include, but are not limited to, intraarterial, intramuscular, intraperitoneal, intraportal, intradermal, epidural, intrathecal administration, or any combination thereof.

In some embodiments, the compounds of the invention may be administered alone (e.g., separately), and/or sequentially, and/or in parallel at the same time (e.g., concurrently), using different administrative routes, but are preferably administered by way of known pharmaceutical formulations, including tablets, capsules or elixirs for oral administration, suppositories for rectal administration, sterile solutions or suspensions for parenteral or intramuscular administration, or via inhalation, and the like. Administration through inhalation is preferably done by using a nebulizer, thus delivering the compound of the invention (e.g., an AT2 receptor agonist or other compound that stimulates an AT2 receptor, or a pharmaceutically acceptable salt, solvate or prodrug thereof) to the small lung tissue including the alveoli and bronchioles, preferably without causing irritation or cough in the treated subject.

Preferably, administration of a therapeutically effective amount of a compound of the invention (or a pharmaceutically acceptable salt, solvate or prodrug thereof) is performed by a combination of administrative routes, either separately (e.g., about 2 or more hours apart from one another), sequentially (e.g., within about 2 hours of one another), or in parallel at the same time (e.g., concurrently), including via inhalation and orally, achieving an effective dosage.

In some embodiments, there is provided a method of treating pulmonary fibrosis, in particular idiopathic pulmonary fibrosis, comprising administering a therapeutically effective amount of a compound of the invention (e.g., an AT2 receptor agonist or other compound that stimulates an AT2 receptor, or a pharmaceutically acceptable salt, solvate or prodrug thereof), through a combination of administrative routes, either separately, sequentially, or in parallel at the same time, preferably via inhalation and orally, in order to achieve effective amount or dosage, to a patient in need of such a therapy.

In some embodiments, there is provided a method of treating pulmonary fibrosis, in particular idiopathic pulmonary fibrosis, comprising administering a therapeutically effective amount of the compound N-Butyloxycarbonyl-3-(4-imidazol-1-ylmethylphenyl)-5-iso-butylthiophene-2-sulfonamide (C21), or a pharmaceutically acceptable salt, solvate or prodrug thereof, through a combination of administrative routes, either separately, sequentially, or in parallel at the same time, preferably via inhalation and orally, in order to achieve effective dosage, to a patient in need of such a therapy.

Such combinations of administrative routes, preferably via inhalation and orally, may be presented as separate formulations of the compound of invention that are optimized for each administrative route.

Such formulations may be prepared in accordance with standard and/or accepted pharmaceutical practice.

According to a further aspect of the invention there is thus provided a pharmaceutical formulation comprising a compound of the invention, in admixture with a pharmaceutically acceptable adjuvant, diluent or carrier, for use in the treatment of idiopathic pulmonary fibrosis.

Compounds of the invention may also be administered in combination with other AT2 agonists that are known in the art, as well as in combination with AT1 receptor antagonists that are known in the art, and/or in combination with an inhibitor of angiotensin converting enzyme (ACE). Non-limiting but illustrative examples of AT₁ receptor antagonists that can be used according to the embodiments include azilsartan, candesartan, eprosartan, fimasartan, irbesartan, losartan, milfasartan, olmesartan, pomisartan, pratosartan, ripiasartan, saprisartan, tasosartan, telmisartan, valsartan and/or combinations thereof. Non-limiting but illustrative examples of ACE inhibitors that can be used according to the embodiments include captopril, zofenopril, enalapril, ramipril, quinapril, perindopril, lisinopril, benazepril, imidapril, trandolapril, fosinopril, moexipril, cilazapril, spirapril, temocapril, alacepril, ceronapril, delepril, moveltipril, and/or combinations thereof.

Compounds of the invention may also be administered in combination with a Galectin-3 inhibitor or established therapies for IPF or fibrosis related diseases that are known in the art, including but not limited to pirfenidone and/or nintedanib.

Such combinations may therefore be useful in the therapeutic treatment of pulmonary fibrosis, in particular IPF.

According to a further aspect of the invention, there is provided a combination product comprising:
(A) an AT2 receptor agonist and/or a compound that stimulates AT2 receptors, or a pharmaceutically acceptable salt, solvate or prodrug thereof, and
(B) an AT1 receptor antagonist, and/or an ACE inhibitor,
   wherein each of components (A) and (B) is formulated in admixture with a pharmaceutically-acceptable adjuvant, diluent or carrier, for use in the therapeutic treatment of pulmonary fibrosis, in particular IPF.

Such combination products provide for the administration of an AT2 receptor agonist and/or a compound that stimulates an AT2 receptor (as defined herein), in conjunction with an AT1 receptor antagonist and/or an ACE inhibitor, and may thus be presented either as separate formulations, wherein at least one of those formulations comprises an AT2 receptor agonist or a compound that stimulates an AT2 receptor (as defined herein, e.g., a compound of the invention, or a pharmaceutically acceptable salt, solvate or prodrug thereof), and at least one formulation comprises an AT1 receptor antagonist and/or an ACE inhibitor, or may be presented (i.e., formulated) as a combined preparation (i.e., presented as a single formulation including an AT2 receptor agonist and/or a compound that stimulates an AT2 receptor together with either an AT1 receptor antagonist or an ACE inhibitor, or both an AT1 receptor antagonist and an ACE inhibitor).

Thus, there is further provided:

(1) a pharmaceutical formulation comprising an AT2 receptor agonist and/or a compound that stimulates an AT2 receptor (e.g., a compound of the invention, or a pharmaceutically acceptable salt, solvate or prodrug thereof) and an AT1 receptor antagonist and/or an ACE inhibitor in admixture with a pharmaceutically-acceptable adjuvant, diluent and/or carrier, for use in the therapeutic treatment of pulmonary fibrosis, in particular idiopathic pulmonary fibrosis; and

(2) a kit of parts comprising components:
(a) a pharmaceutical formulation comprising an AT2 receptor agonist and/or a compound capable of stimulating AT2 receptors (e.g., a compound of the invention, or a pharmaceutically acceptable salt, solvate or prodrug thereof), in admixture with a pharmaceutically-acceptable adjuvant, diluent and/or carrier; and
(b) a pharmaceutical formulation including an AT1 receptor antagonist, and/or an ACE inhibitor, in admixture with a pharmaceutically-acceptable adjuvant, diluent and/or carrier,
   which components (a) and (b) are each provided in a form that is suitable for administration in conjunction with the other, for use in the therapeutic treatment of pulmonary fibrosis, in particular idiopathic pulmonary fibrosis.

Depending upon the patient to be treated and the route of administration, the compounds of the invention may be administered at varying doses. Although doses will vary from patient to patient, suitable daily doses are in the range of about 0.1 to about 1000 mg (e.g., 0.1, 0.5, 1, 2, 5, 10, 15, 20, 25, 30, 35, 40, 45, 50, 75, 100, 150, 200, 250, 300, 350, 400, 450, 500, 550, 600, 650, 700, 750, 800, 850, 900, 950, 1000 mg, and the like, or any range or value therein) per patient, administered in single or multiple doses. More preferred daily doses are in the range of about 0.1 to about 250 mg (e.g, 0.2, 0.3, 0.4, 0.5, 1, 1.5, 2, 2.5, 3, 3.5, 4 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250 mg, and the like, or any range or value therein) per patient. A particular preferred daily dose is in the range of from about 0.3 to about 100 mg per patient.

Individual doses of compounds of the invention may be in the range 0.1 to 100 mg (e.g., 0.3, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 mg, and the like, or any range or values therein).

In any event, the physician, or the skilled person, will be able to determine the actual dosage, which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this invention.

The benefits of using the compounds of the invention, preferably via a combination of administrative routes, separately, and/or sequentially, and/or in parallel at the same time is to produce a tailored treatment for the patient in need of the therapy, with the possibility of preventing and/or reducing side effects, and also tune the correct dosage levels of a therapeutically effective amount of a compound of the invention.

The compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties than compounds known in the prior art. Such effects may be evaluated clinically, objectively and/or subjectively by a health care professional, a treatment subject or an observer.

Subjects suitable to be treated with formulations of the present invention include, but are not limited to, mammalian subjects. In some embodiments, the subject can be a human subject.

The usability of the compounds of the invention for the therapeutic treatment of pulmonary fibrosis, in particular IPF, finds support from the following aspects:

Compounds of the invention have an anti-fibrotic effect, with reduction of fibrosis and prevention of further deposition of extra cellular matrix. Compounds of the invention affect wound healing in a proper way and also have an anti-apoptotic effect, thereby preventing apoptosis for alveolar endothelial cells, which is an initiating factor for the development of pulmonary fibrosis. Compounds of the invention also have an anti-proliferative effect, thus reducing the cancer-like proliferation of fibroblasts and myofibroblasts in pulmonary fibrosis. Compounds of the invention also improve vascular remodelling in pulmonary fibrosis, thereby reducing secondary pulmonary hypertension. Finally, compounds of the invention demonstrate anti-inflammatory and anti-cytokine effects.

### EXAMPLES

### Example 1

Patients have a diagnosis of Idiopathic Pulmonary Fibrosis, IPF, who generally are above the age of 60 years, often with concomitant diseases, like ischemic heart disease, diabetes etc. Patients may either be treated for IPF with currently established drugs, e.g. pirfenidone and/or nintedanib or treatment-naïve. Each patient receives a compound of the invention, C21, in a dose range between 0.3 mg and 100 mg daily that is administered once or several times daily. Control groups are administered placebo, or one or more currently established therapies, e.g. pirfenidone and/or nintedanib. The compound, C21, is in a formulation hat can be administered to the patient orally, via inhalation, intravenously or as a combination of more than one route of administration for the treatment of IPF. The treatment is evaluated through efficacy variables such as objective measurements of improved lung capacity/cardiac function and subjective symptoms and quality of life—both at visits to doctors' office and in the patient's home or work environment. Beneficial effects are recorded and followed after one to several months' treatment with the compound, C21, vs placebo or vs currently established therapy(-ies), e.g. pirfenidone and/or nintedanib.

### Example 2

Patients have a diagnosis of Idiopathic Pulmonary Fibrosis, IPF, and are treated according to EXAMPLE 1. Objective measures showing beneficial effects include improvement of lung function tests (such as Forced Vital Capacity, FVC) using e.g. spirometry, or demonstrated by an improvement of relevant biomarkers, e.g. selected cytokines, selected serum proteins, selected neo-epitopes, selected blood cells, genomic markers, telomere length and functional markers. Furthermore, cardiac function tests, e.g. echo or MRI, may indicate improvements in the underlying lung disease resulting in improved cardiac performance. Benefits may also be seen as an improvement in progression-free survival, where the number of deaths is analyzed together with the number of patients presenting with pre-defined marked reduction of FVC and/or exercise capacity. Benefits can be either self-reported or objectively recorded. Improvements in symptoms may include less dyspnea, less cough, less fatigue, increased exercise capacity and improved quality of life. Improvements may be seen in treatment-naïve patients as well as in those already on treatment with established therapies for IPF. A few weeks after starting treatment in a patient with IPF with a compound of the invention, C21, blood tests reveal an improvement in biomarkers that are relevant for the progression of the disease. The patient reports a slight improvement in dyspnea and cough without any deterioration of exercise capacity. A prolonged treatment with C21 will give improvements in objective measurements of lung function as well as of cardiac parameters, indicating improvements of the underlying progressive disease.

1. A method of treating pulmonary fibrosis in a subject in need thereof, comprising:
administering to the subject a therapeutically effective amount of a composition comprising an angiotensin II receptor agonist or a pharmaceutically acceptable salt, solvate or prodrug thereof.

2. (canceled)

3. The method according to claim 1, wherein the angiotensin II receptor agonist is a selective agonist of an angiotensin II type 2 (AT2) receptor or a pharmaceutically acceptable salt, solvate or prodrug thereof.

4. The method according to claim 3, wherein selective agonist of an angiotensin II type 2 (AT2) receptor is a non-peptide selective agonist or a pharmaceutically acceptable salt, solvate or prodrug thereof.

5. The method according to claim 1, wherein the angiotensin II receptor agonist is N-Butyloxycarbonyl-3-(4-imidazol-1-ylmethylphenyl)-5-iso-butylthiophene-2-sulfonamide (C21) or a pharmaceutically acceptable salt, solvate or prodrug thereof.

6. The method according to claim 1, wherein the pulmonary fibrosis is idiopathic pulmonary fibrosis.

7. The method according to claim 1, wherein the administering comprises introducing the composition into the subject nasally, orally, parenterally or by inhalation.

8. The method according to claim 1, wherein the composition is administered separately, sequentially and/or concurrently by more than one administrative route.

9. The method according to claim 8, wherein the combination of administrative routes is inhalation and oral.

10. The method according to claim 1, wherein the composition is administered to a subject at a daily dose in the range of from about 0.3 to about 100 mg.

11. The method according to claim 1, wherein the composition is administered in combination with an AT1 receptor antagonist.

12. The method according to claim 11, wherein the AT1 receptor antagonist is selected from the group consisting of losartan, azilsartan, candesartan, eprosartan, fimasartan, irbesartan, milfasartan, olmesartan, pomisartan, pratosartan, ripiasartan, saprisartan, tasosartan, telmisartan, valsartan and combinations thereof.

13. The method according to claim 11, wherein the composition and the AT1 receptor antagonist are administered separately, sequentially and/or concurrently.

14. The method according to claim 11, wherein the AT1 receptor antagonist is provided in the same composition as or in a separate composition from the composition comprising the angiotensin II receptor agonist.

15. The method according to claim 1, wherein the composition is administered in combination with an inhibitor of angiotensin converting enzyme (ACE).

16. The method according to claim 15, wherein the angiotensin converting enzyme (ACE) inhibitor is selected from the group consisting of captopril, zofenopril, enalapril, ramipril, quinapril, perindopril, lisinopril, benazepril, imidapril, trandolapril, fosinopril, moexipril, cilazapril, spirapril, temocapril, alacepril, ceronapril, delepril, moveltipril, and combinations thereof.

17. The method according to claim 15, wherein the composition and the angiotensin converting enzyme (ACE) inhibitor are administered separately, sequentially and/or concurrently.

18. The method according to claim 15, wherein the inhibitor of angiotensin converting enzyme (ACE) is provided in the same composition as or in a separate composition from the composition comprising the angiotensin II receptor agonist.

19. The method according to claim 1, wherein the composition is administered in combination with an AT1 receptor antagonist and an angiotensin converting enzyme (ACE) inhibitor.

20. The method according to claim 19, wherein the composition, the AT1 receptor antagonist and the angiotensin converting enzyme (ACE) inhibitor are administered separately, sequentially and/or concurrently to one another.

21. The method according to claim 19, wherein the AT1 receptor antagonist and the angiotensin converting enzyme (ACE) inhibitor are provided in the same composition as the composition comprising the angiotensin II receptor agonist.

22. The method according to claim 20, wherein the AT1 receptor antagonist and the angiotensin converting enzyme (ACE) inhibitor are provided in separate compositions from one another and/or from the composition comprising the angiotensin II receptor agonist.

23. The method according to claim 1, wherein the composition is administered in combination with a Galectin-3 inhibitor or one or more established therapies for IPF or a fibrosis related disease.
